(19) United States
(12) Patent Application Publication
(10) Pub. No.: US 2003/0158593 A1
(43) Pub. Date: Aug. 21, 2003

(54) Cardiac garment

(76) Inventors: Marlin S. Heilman, Sarver, PA (US); Emil D. Oskin, Natrona Heights, PA (US); Philip C. Skalos, Pittsburgh, PA (US)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/364,374

(22) Filed: Feb. 12, 2003

Related U.S. Application Data
(60) Provisional application No. 60/357,759, filed on Feb. 19, 2002.

(51) Int. Cl. A61N 1/18

(52) U.S. Cl. 607/149

(57) ABSTRACT

A support garment for an external wearable defibrillator provides support for and proper positioning of monitoring electrodes and/or therapy electrodes and associated cabling, and can require less than 1.5 pounds of tension to maintain contact between the monitoring electrodes and the patient's skin. The garment may be fabricated in a range of sizes, provide for customized fitting to each patient, and can be made from a washable material that is comfortable for long-term wear and which incorporates electrically conductive cloth to provide a more comfortable electrical connection between the therapy electrodes and the patient's skin.

### RELATED APPLICATIONS

This application claims priority to U.S. Provisional Patent Application Serial No. 60/357,759, filed Feb. 19, 2002.

### BACKGROUND

This invention relates to an improved garment for comfortably and operatively holding a wearable external defibrillator, and particularly associated electrodes, in contact with a patient to permit the application of electric therapy to the heart muscle by the defibrillator if a treatable arrhythmia is detected.

Technology is available for correcting excessively slow heart rates (bradycardia) using implantable devices, commonly referred to as pacemakers, which deliver microjoule electrical pulses to a slowly beating heart in order to speed the heart rate up to an acceptable level. Also, it is well known to deliver high energy shocks (e.g., 180 to 360 joules) via external paddles applied to the chest wall in order to correct excessively fast heart rates, and prevent the possible fatal outcome of ventricular fibrillation or certain ventricular tachycardias. Bradycardia, ventricular fibrillation, and ventricular tachycardia are all electrical malfunctions (arrhythmias) of the heart. Each may lead to death within minutes unless corrected by the appropriate electrical stimulation.

One of the most deadly forms of heart arrhythmias is ventricular fibrillation, which occurs when the normal, regular electrical impulses are replaced by irregular and rapid impulses, causing the heart muscle to stop normal contractions and to begin to quiver. Normal blood flow ceases, and organ damage or death may result in minutes if normal heart contractions are not restored. Although frequently not noticeable to the victim, ventricular fibrillation is often preceded by ventricular tachycardia, which is a regular but fast rhythm of the heart. Because the victim has no noticeable warning of the impending fibrillation, death often occurs before the necessary medical assistance can arrive.

Because time delays in applying the corrective electrical treatment may result in death, implantable pacemakers and defibrillators have significantly improved the ability to treat these otherwise life threatening conditions. Being implanted within the patient, the device continuously monitors the patient's heart for treatable arrhythmias and when such is detected, the device applies corrective electrical pulses directly to the heart.

Normal heart function often can be restored to a person suffering ventricular fibrillation or ventricular tachycardia by a procedure known as cardioversion, the synchronized application of electric therapy to the heart muscle. Pacemakers and defibrillators that apply corrective electrical pulses externally to the patient's chest wall also are used to correct such life-threatening arrhythmias but suffer from a drawback insofar as it may not be possible to apply the device in time during an acute arrhythmic emergency to save the patient's life. Such treatment is needed within a few minutes to be effective.

Consequently, when a patient is deemed at high risk of death from such arrhythmias, electrical devices often are implanted so as to be readily available when treatment is needed. Alternatively, such patients are kept in a hospital where corrective electrical therapy is generally close at hand. Long term hospitalization, however, is frequently impractical due to its high cost, or due to the need for patients to engage in normal daily activities.

Non-adhesive electrodes, used as either ECG sensing electrodes or treatment electrodes, can have significant advantages when compared to adhesive electrodes. However, one of the challenges associated with using non-adhesive electrodes is maintaining contact between the electrode and the patient's skin. This problem is complicated by the wide variation in patient sizes and shapes, and by the changes in size and shape that occur with movement.

Some of the problems have been addressed in U.S. Design Pat. No. 6,065,154, assigned to the assignee hereof, which is hereby incorporated by reference. A wearable external defibrillator is disclosed in U.S. Pat. No. 5,741,306 assigned to the assignee hereof, which is hereby incorporated herein by reference. However, there remain some disadvantages with adaptability to different sizes and shapes of patients and with comfortably maintaining good electrical contact between the electrodes and the patient's skin, which are addressed by the improved cardiac garment described hereinafter.

### SUMMARY

According to the invention, a support garment for an external wearable defibrillator can provide support for and proper positioning of ECG monitoring electrodes and/or therapy electrodes and associated cabling. The garment can have several main components including a garment back and a detachable garment strap. The garment back can include a rear therapy electrode pocket and a pair of over-the-shoulder straps. Each over-the-shoulder strap can encircle an adjacent shoulder of the patient, like the straps on a typical back-pack, in a figure-eight shape which can help reduce the effects of patient movement and ensure better skin contact of the therapy electrodes. The garment strap can include and elastic belt member which can be slidably attached to an organizer plate which can hold the monitoring electrodes against the patient's skin. The garment strap can also include front therapy electrode pocket which the elastic belt member holds against the patient's skin. The garment back can also include a support strap which extends between the front therapy electrode pocket and one of the over-the-shoulder straps to help prevent downward vertical motion of the front therapy electrode pocket. The support garment can require less than 1.5 pounds of tension to maintain contact between the electrodes and the patient's skin. The treatment electrode pockets made using an electrically conductive cloth to provide a path of electrical conduction between the therapy electrodes and the patient's body while also providing a more comfortable interface with the patient's skin. The garment may be fabricated in a range of sizes, can provide for adjustment during the process of fitting to a particular patient, and can be made from a washable material that is comfortable for long-term wear.

Other details, objects, and advantages of the invention will become apparent from the following detailed description and the accompanying drawings figures of certain embodiments thereof.

### BRIEF DESCRIPTION OF THE DRAWING FIGURES

A more complete understanding of the invention can be obtained by considering the following detailed description in conjunction with the accompanying drawings, in which:

FIG. 1a is an illustration of an embodiment of the cardiac garment as worn on the front of the body.

FIG. 1b is an illustration of an embodiment of the cardiac garment as worn on the rear of the body.

FIG. 2a is a plan view of the cardiac garment back, on-body side.

FIG. 2b is a plan view of the off-body side of the cardiac garment back.

FIG. 3a is a plan view of a strap for the cardiac garment, off-body side.

FIG. 3ba is a plan view of the on-body side of the strap.

FIG. 4a is a perspective view of an embodiment of an organizer plate.

FIG. 4b side view of the organizer plate.

FIG. 5a is a plan view, off-body side, of the organizer plate and installed on the garment strap with an electrode assembly attached to the organizer plate.

FIG. 5b is a plan view, on-body side, of the organizer plate shown in FIG. 5a.

### DETAILED DESCRIPTION OF CERTAIN EMBODIMENTS

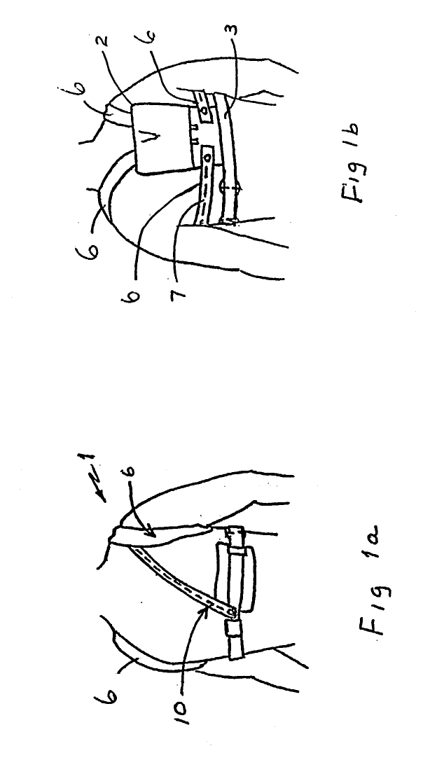

Referring now to the drawing figures, wherein like reference numbers refer to similar parts throughout the several views, an embodiment of a support garment 1 for a patient worn external monitoring and energy delivery apparatus is shown in FIGS. 1a and 1b secured around the chest of a patient. The garment 1 can have several main components including a garment back 2 and a fixed or removable garment strap 3. The garment back 2 can be secured around the upper torso, e.g., the chest, of the patient by a pair of over-the-shoulder straps 6 and by the garment strap 3. Particularly, an upper portion of the garment back 2 can be secured around the chest of the patient by the over-the-shoulder straps 6 whereas the garment strap 3 can secure a lower portion of the garment back 2 around the lower torso, e.g., the rib cage, of the patient. The garment back 2 and the garment strap 3 can each be provided in a range of different sizes to permit custom fitting to each patient. For example, at least two sizes of the garment back 2 and about eight sizes of the garment strap 3 are contemplated to fit patients with a rib cage circumference of 26 to 54 inches. The range of component sizes may be altered according to the elastic capabilities of the material used. Also, the number of fixed sizes provided may be reduced through the addition of adjustment features.

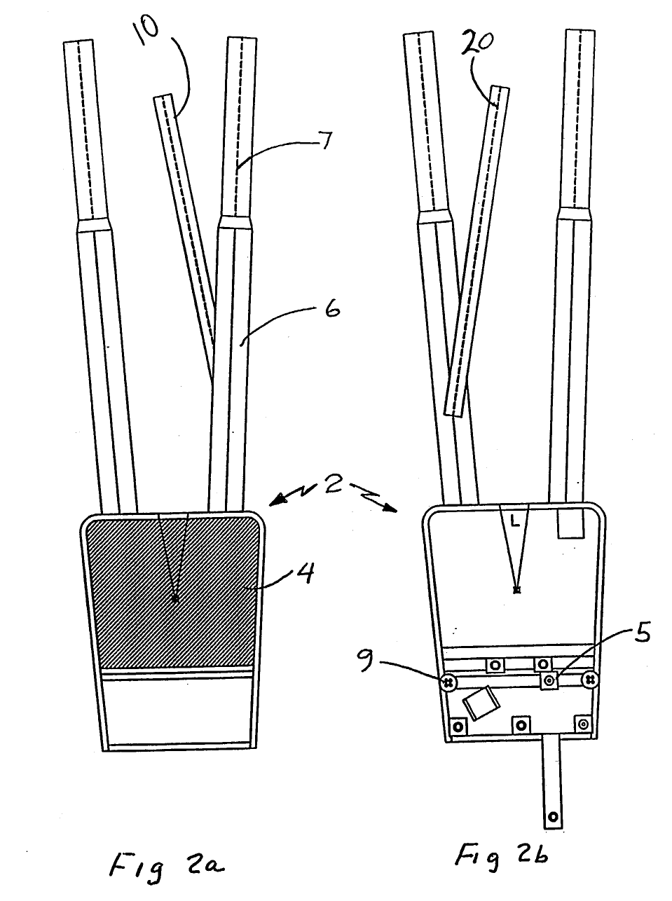

FIGS. 2a and 2b show the garment back 2 in more detail. The garment back 2 can be constructed from an open weave elasticized fabric and can include a rear treatment electrode pocket 4 which can be made of a conductive metal cloth, such as a metal mesh cloth. For example, silver bearing cloth mesh (silver impregnated cloth) can be used for at least part of the pocket 4 as the interface between one or more therapy electrodes (not shown) held in the pocket 4 and the patient's skin. The silver bearing cloth mesh can provide a path of electrical conduction to allow on-skin detection for the therapy electrodes while also providing a more comfortable interface with the patient's skin. Other types of suitable conductive cloth can be made from, for example, the following materials—carbon fiber, copper-sulfide coated fiber, silver metalized fabric, nickel-silver metalized fabric, silver-copper metalized fabric, gold-nickel-copper metalized fabric, copper-indium metalized fabric, copper-gold metalized fabric, and cloth made from metallic threads. These materials may be woven, non-woven, plated or coated onto the fabric. Some Common fabrics can include, for example, nylon, polyester, cotton, nylon tricot knit, and elastomeric foams.

Snaps 5 can be used to hold electrode belt components and the garment strap 3 onto the garment back 2. The over-the-shoulder straps 6 can be constructed of padded soft fabric and an elastic strap, which can be provided with multiple button holes 7 to allow adjustment while fitting patients. After securing the elastic straps 6 over the patients shoulders by inserting buttons 9 through button holes 7, any excess strap 6 material may be cut off to prevent loosening of the garment 1 by the patient. Other methods of attaching and fixing the straps 6 may be also provided, for example snaps, Velcro, plastic rivets, buckles, and the like. Each over-the-shoulder strap 6 can encircle an adjacent shoulder of-the patient, like the straps on a typical back-pack, in a figure-eight shape which can help reduce the effects of patient movement and ensure better skin contact of the therapy electrodes. Specifically, each of the pair of over-the-shoulder straps 6 can have one end connected to an upper portion at one side of the garment back 2 and one free end. The free end can be wrapped over the patient's shoulder and thence under the patient's arm on that side, and the free end then attached to the garment back 2 via button holes 7 and buttons 9 in a figure-eight configuration. Although shown as non-removably attached to the top of the garment back 2, it should be understood that the ends of the strap 6 opposite the “free ends” referred to above could be instead also be removably attached to the garment back 2, such as in the same type of manner as the free ends are attached. The figure-eight design can reduce the effects of patient movement and position and ensures better skin contact of the garment back 2 than “suspender” type configurations.

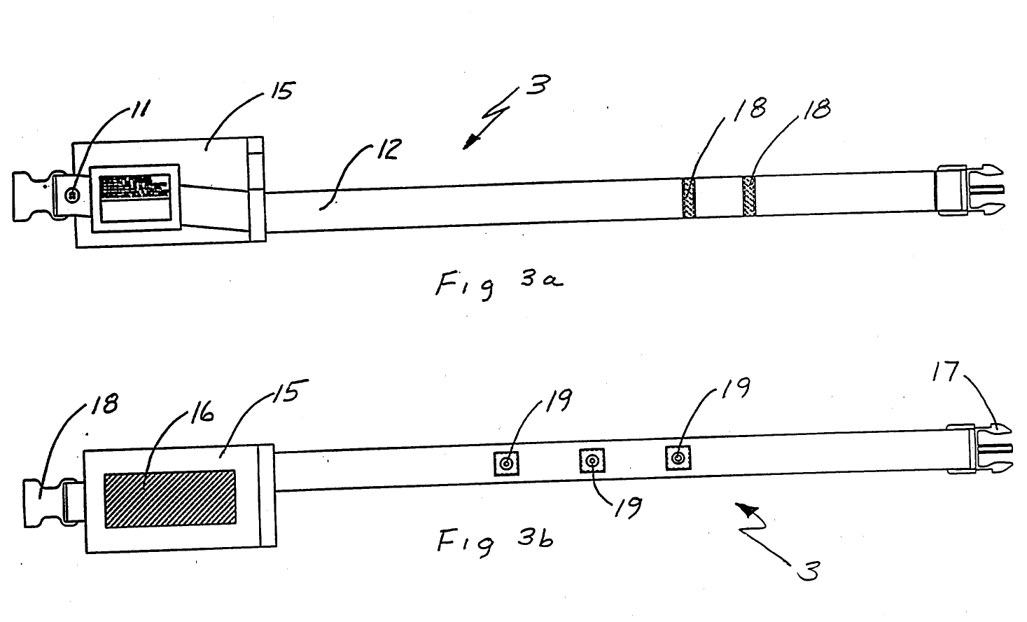

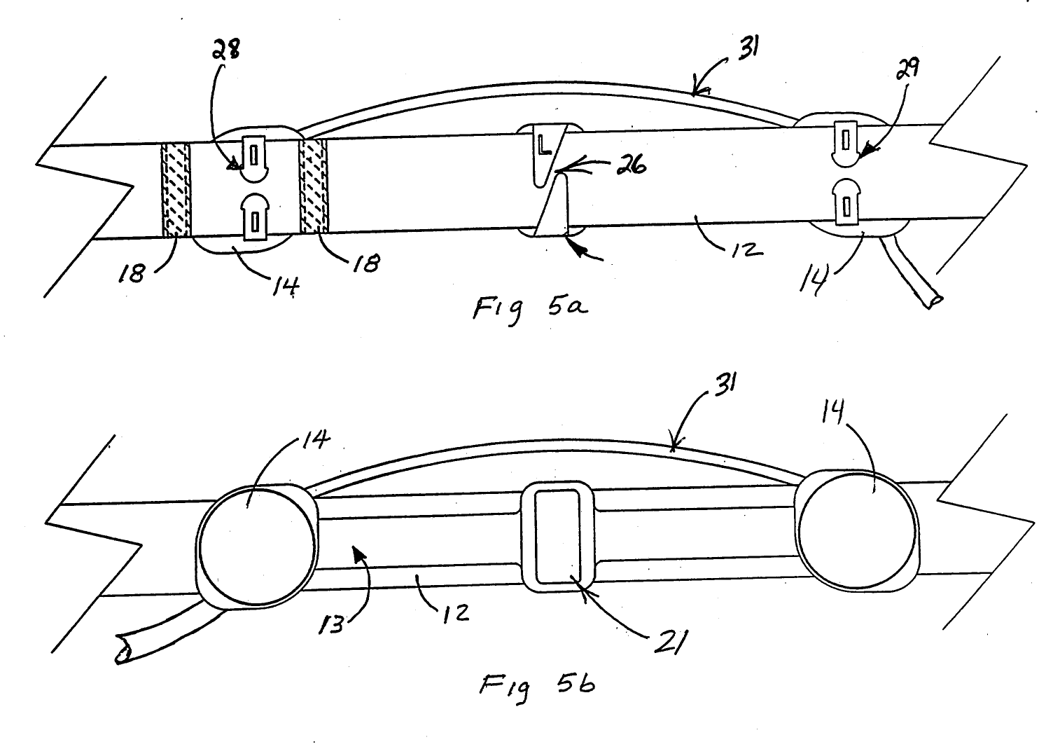

FIG. 3a and FIG. 3b show each side of the garment strap 3, which can be comprised mainly of an elastic belt 12 which can hold other components of the garment 1 against the body of the patient. In particular, an organizer plate 13, which can include monitoring electrodes 14, (see FIGS. 5a and 5b) and a front therapy electrode pocket 15 can be held against the patient's skin by the elastic belt 12. The garment back 2 can further include a strap 10 associated with the front therapy electrode pocket 15 (see FIGS. 2a and 2b) which extends between the front therapy electrode pocket 15 and one of the over-the-shoulder straps 6, for example the strap 6 on the patient's right side as shown in FIG. 1a. Button holes 20 can be provided in the therapy electrode pocket strap 10 so that, when the patient is fitted, the therapy electrode pocket strap 10 can be secured to the front therapy electrode pocket 15 via a button 11. The front therapy electrode pocket strap 10 with button holes 20 can help prevent downward vertical motion of the front therapy electrode pocket 15. Additionally, the connections on the garment back 2, as well as each of the connectable portions of, for example, the therapy electrode pocket strap 10, the over-the-shoulder straps 6, and the garment strap 3 can be designed to connect together in a manner unique to each connection member, for example using reversed snaps. This can be done to reduce the potential for misassembly of the various members when the garment 1 is fitted to the patient.

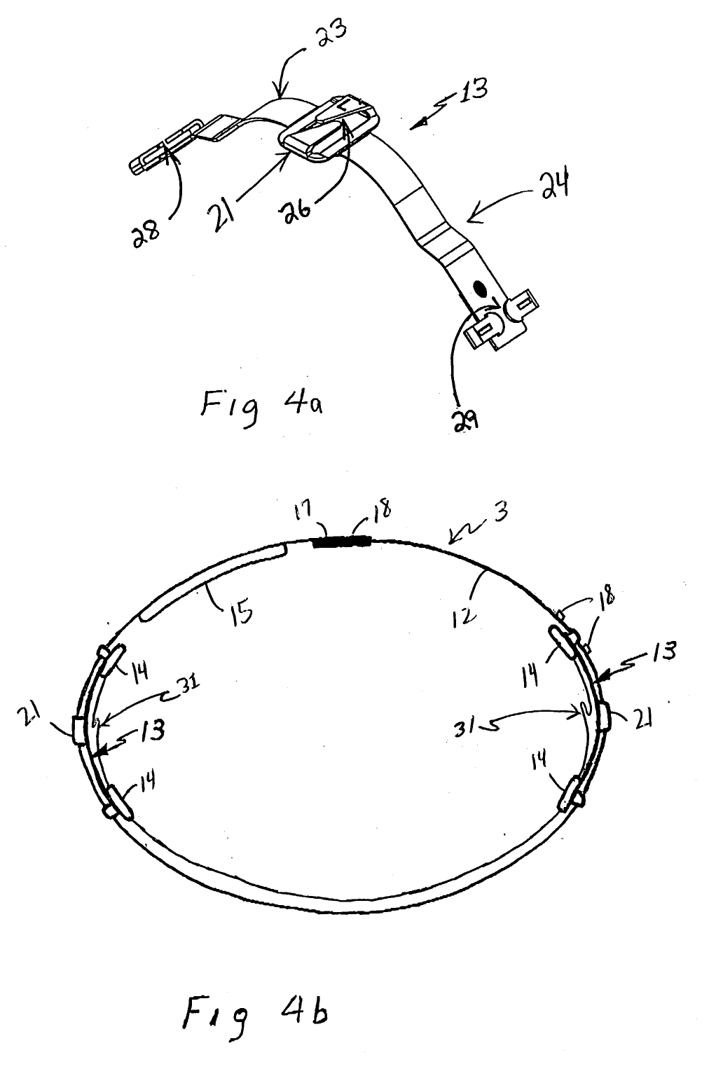

FIG. 4a illustrates an embodiment of an organizer plate 13, which can be slidably attached to the elastic belt 12. The organizer plate 13 can be manufactured from a flexible plastic material. The organizer plate 13 can be have a central portion 21, and curved arm portions 23, 24 extending out from each side of the central portion 21. The curved arm portions 23, 24 can be flexible and can be pre-formed with a curvature such as shown best in FIG. 4b. A pair of clip members 26 can be provided on the central portion 21 of the organizer plate 13 which permit the elastic belt strap 12 to be slidably secured to the central portion 21 of the organizer plate 13. Additional clip pairs 28, 29 can be provided at either end of curved arm portions 23, 24 of the organizer plate 13 for further slidably attaching the elastic belt strap 12 to the curved arm portions 23, 24. An electrode harness assembly 31 can be attached to the organizer plate 13 on the ends of the curved arm portions 23, 24, on opposite sides of the clip pairs 28, 29. The electrode harness assembly 31 can include the monitoring electrodes 14 (see FIGS. 5a and 5b) mentioned previously. The electrode harness assembly 31 can include four individual monitoring electrodes 14 connected to two separate organizer plates 13, as shown in FIG. 4b. The organizer plates 13 can be produced in several different sizes for custom fitting to different patients. For example, the electrode harness assembly 31 can be produced in a single size which can be designed, in regard to spacing between both the individual electrode 14 pairs and between the electrode 14 pairs attached to each organizer plate 13, to be used with several different organizer plate 13 sizes. For example, the organizer plates 13 can be produced in sizes having 4, 6, and 8 inches between centers where the individual electrodes 14 are attached to the clip pairs 28, 29. Thus, different size patients can be easily fitted using a single, standard sized electrode harness assembly 31 and variably sized organizer plates 13. The organizer plate 13 can be designed to transfer force from the elastic belt strap 12 to the monitoring electrodes 14 to maintain contact between the patient's body and the monitoring electrodes 14. In particular, the elastic belt strap 12, when tightened around the rib cage of the patient, will draw the central portion 21 of the organizer plate toward the patient's body. The force exerted by the elastic belt strap 12 on the central portion 21 will be transferred via the curved arm portions 22, 23 to the monitoring electrodes 14 attached to the ends thereof. Consequently, the monitoring electrodes 14 will be urged against the body of the patient thereby facilitating good electrical contact therebetween. The elastic belt 12 can have a low spring rate, and the garment strap 3 can be designed to require less than 1.5 pounds of tension exerted by the elastic belt 12 to maintain contact between the patient's skin and the monitoring 14 electrodes.

FIG. 4b shows how the garment strap 3 can hold the front therapy electrode pocket 15 and a pair of organizer plates 13 containing the ECG monitoring electrodes 14 against the patient's skin. The front therapy electrode pocket 15 can be designed with a window made of silver bearing cloth mesh 16. The silver bearing cloth mesh 16 can provide a path of electrical conduction to allow on-skin detection for the therapy electrodes. Male 17 and female belt buckle 18 ends can be assembled onto the ends of the elastic belt strap 12 to facilitate donning the garment 1. Two stops 18 made of thick plush cloth can be sewn onto the elastic belt strap 12. The stops 18 can help ensure proper positioning of the monitoring electrodes 14. The garment strap 3 can be assembled to the garment back 2 with three snaps 19. One of the snaps 19 can be reversed relative to the other two snaps 19. This snap arrangement can help prevent accidental improper assembly of the garment strap 3 to the garment back 2. Other methods of guiding the assembly of the garment components and electrodes may be employed, for example color coding, shape differentiation, labels, and the like.

FIGS. 5a and 5b are enlarged views showing one organizer plate 13 with monitoring electrodes 14 slidably installed onto the garment strap 3. Multiple sizes of the organizer plates 13 can be provided to provide a wider range of adjustment of the elastic belt strap 12. The organizer plate 13 can be designed to allow the elastic belt strap 12 to slide freely, within a limited range, via multiple clip pairs 26, 28 and 29 while maintaining tension around the patient as described previously. The organizer plates 13 in combination with the stops 18 help limit the sliding motion of the monitoring electrodes 14 on the patient's body. Some sliding motion of the organizer plates 13 along the elastic belt 12 is permitted, but the range of the motion is limited by the stops 18 on one side of the elastic belt 12 and the front electrode pocket 15 on the other side. Additional stops 18 could also be used on the other side of the elastic belt 12, either alone or in conjunction with the front electrode pocket 15. Providing a limited range of sliding movement permits the garment 1 and the monitoring electrodes 14 to better conform to the patient's body in a variety of positions while maintaining the desired location of the monitoring electrodes 14.

Although certain embodiments of the invention have been described in detail, it will be appreciated by those skilled in the art that various modifications to those details could be developed in light of the overall teaching of the disclosure. Accordingly, the particular embodiments disclosed herein are intended to be illustrative only and not limiting to the scope of the invention which should be awarded the full breadth of the following claims and any and all embodiments thereof.

What is claimed is:

1. A two-part support garment for a patient worn monitoring and energy delivery apparatus, the support garment comprising:
a. a garment back having a pair of over-the-shoulder straps adapted to secure an upper portion of said garment back around the upper torso of the patient when said support garment is donned by the patient; and
b. a garment strap attachable to said garment back and adapted to secure a lower portion of said garment back around the lower torso of the patient.

2. The support garment of claim 1 further comprising said pair of over-the-shoulder straps configured to encircle respective left and right shoulders of the patient such that said garment back is carried in a figure-eight configuration.

3. The support garment of claim 1 further comprising:
a. said garment back having a first therapy electrode pocket; and
b. said first therapy electrode pocket having an electrically conductive cloth portion which is held adjacent the patient's skin when said support garment is donned by the patient.

4. The support garment of claim 3 wherein said electrically conductive cloth portion comprises a metal mesh cloth portion.

5. The support garment of claim 4 wherein said metal mesh cloth portion is comprised at least partially of silver.

6. The support garment of claim 3 further comprising:
a. said garment strap having a second therapy electrode pocket; and
b. said second therapy electrode pocket having an electrically conductive cloth portion which is held adjacent the patient's skin when said support garment is donned by the patient.

7. The support garment of claim 6 further comprising a support strap attachable between said second therapy electrode pocket and one of said pair of over-the-shoulder straps.

8. The support garment of claim 1 wherein at least one of said garment strap and said over-the-shoulder straps are attachable to said garment back in a unique manner to inhibit misassembly.

9. The support garment of claim 1 wherein said garment strap further comprises:
a. a belt portion;
b. at least one monitoring electrode; and
c. an organizer plate attachable to said belt portion, said at least one monitoring electrode attachable to said organizer plate, said organizer plate transferring force from said belt portion to said at least one monitoring electrode to hold said at least one monitoring electrode against the patient's skin.

10. The support garment of claim 9 wherein as little as about 1.5 pounds of tension need be exerted by said belt portion to maintain contact between said at least one monitoring electrode and the body of the patient.

11. The support garment of claim 9 further comprising:
a. said organizer plate slidably attachable to said belt portion; and
b. said belt portion having spaced apart stops which define a limited range of sliding movement of said organizer plate relative to said belt portion.

12. The support garment of claim 1 further comprising a support strap attachable between at least one of said pair of over-the-shoulder straps and said second therapy electrode pocket; and

13. The support garment of claim 1 wherein different sizes of said garment back are attachable to different sizes of said garment strap.

14. A support garment for a patient worn monitoring and energy delivery apparatus, the support garment comprising:
a. a garment strap adapted to at least partially secure said support garment around the torso of the patient;
b. at least one monitoring electrode; and
c. an organizer plate slidably attachable to said garment strap, said at least one monitoring electrode attachable to said organizer plate, said organizer plate transferring force from said garment strap to said at least one monitoring electrode to hold said at least one monitoring electrode against the patient's skin.

15. The support garment of claim 14 further comprising:
a. said garment strap having a belt portion;
b. said organizer plate slidably attachable to said belt portion; and
c. said belt portion having spaced apart stops which define a limited range of sliding movement of said organizer plate relative to said belt portion.

16. The support garment of claim 15 wherein only about 1.5 pounds of tension need be exerted by said belt portion to maintain contact between said at least one monitoring electrode and the patient's skin.

17. The support garment of claim 15 further comprising:
a. a second organizer plate slidably attached to said belt portion;
b. a therapy electrode pocket attached to said belt portion intermediate and end of said belt portion and said second organizer plate; and
c. said therapy electrode pocket limiting a range of sliding movement of said second organizer plate relative to said belt portion.

18. A support garment for a patient worn monitoring and energy delivery apparatus, the support garment comprising at least one therapy electrode pocket having an electrically conductive cloth portion which is held adjacent the patient's skin when said support garment is donned.

19. The support garment of claim 18 wherein said electrically conductive cloth portion comprises a metal mesh cloth portion.

20. The support garment of claim 19 wherein said metal mesh cloth portion is comprised at least partially of silver.
